(19) United States
(12) Patent Application Publication
(10) Pub. No.: US 2010/0028990 A1
(43) Pub. Date: Feb. 4, 2010

(54) STERILE BIOREACTOR BAG WITH INTEGRATED DRIVE UNIT

(75) Inventors: Scott T. Broadley, Los Angeles, CA (US); Patricia R. Benton, Foothill Ranch, CA (US)

Correspondence Address: KNOBBE MARTENS OLSON & BEAR LLP, 2040 MAIN STREET, FOURTEENTH FLOOR, IRVINE, CA, 92614, US

(73) Assignee: BROADLEY-JAMES CORPORATION, Irvine, CA (US)

(21) Appl. No.: 12/525,298

(22) PCT Filed: Feb. 14, 2008
(86) PCT No.: PCT/US08/54022

§ 371 Date: Jul. 30, 2009

Related U.S. Application Data
(60) Provisional application No. 60/890,145, filed on Feb. 15, 2007.

(51) Int. Cl. C12M 1/12

(52) U.S. Cl. 435/289.1

(57) ABSTRACT

Flexible, sterilizable, disposable bioreactors are provided with integrated fluidic drive units that agitate media inside the bioreactor without introducing contamination. The bioreactor system (20) includes a flexible bag (202) with a fluid activated drive unit (204) in sealed cooperation with the bag (202).

### BACKGROUND OF THE INVENTION

1. Field of the Invention

This application relates to a bioreactor system for use in culturing cells. More particularly, this application relates to a flexible, disposable bioreactor bag having an integrated drive unit for introducing agitation in a sterile manner.

2. Description of the Related Art

Bioreactors (also referred to as fermenters) include containers used for fermentation, enzymatic reactions, cell culture, tissue engineering, and food production, as well as in the manufacture of biologicals, chemicals, biopharmaceuticals, microorganisms, plant metabolites, and the like. Bioreactors vary in size from benchtop fermenters to large stand-alone units of various sizes. The stringent asepsis requirements for sterile production in some bioreactors can require elaborate systems to achieve the desired product volumes. Consequently, the production of products in aseptic bioreactors can be costly which provides the motivation for pursuing improved systems.

The expense of producing cells, biopharmaceuticals, biologicals and the like in aseptic bioreactors is exacerbated by the required cleaning, sterilization and validation of the standard bioreactors (e.g., stainless steel or glass containers). Attempts have been made to solve this problem with the development of pre-sterilized disposable bioreactor systems that need not be cleaned, sterilized or validated by end users.

Some companies have developed a sterilizable disposable single use bioreactor (referred to herein as a “bioreactor bag”) that do not require cleaning or sterilizing before each use. Such bioreactors are made from sheets of flexible material which is configured to form a bag. The bag is partially filled with media and then inflated with air that continually passes through the bag's headspace. The media is mixed and aerated by rocking the bags to increase the air-liquid interface. However, since there is typically no solid housing supporting the bags, the bags may become cumbersome and difficult to handle as they increase in size. Furthermore, the wave action within the rocking bag can create damaging turbulent forces. Certain cell cultures, particularly human cell cultures, may benefit from more gentle conditions.

Other companies have developed flexible bioreactor bags with a rotational assembly that attaches to the shaft of a separate drive motor (e.g., an electrically driven drive motor disposed outside of the bag). In some embodiments, one or more impellers are coupled to the rotational assembly inside the bag, allowing the media to be stirred in a manner simulating the hydrodynamic environment of larger, non-disposable bioreactor systems. However, often such configurations may introduce contamination into the bag through, for example, bearings on the rotational means, or another interaction between the un-sterile external drive shaft and the bioreactor bag. Another disadvantage of these systems is the requirement for an external motor to drive the rotational assembly, creating another point of maintenance and expense. Furthermore, the design of these systems can be difficult to scale down to a development-scale reactor.

### SUMMARY OF CERTAIN EMBODIMENTS

The system, method, and devices of the invention each have several aspects, no single one of which is solely responsible for its desirable attributes. Without limiting the scope of this invention, its more prominent features will now be discussed briefly. After considering this discussion, and particularly after reading the section entitled “Detailed Description of Certain Embodiments” one will understand how the features of this invention provide advantages over other bioreactor systems.

In one aspect of the invention, a bioreactor system includes a flexible bag and a fluid activated drive unit in sealed cooperation with the bag. In an embodiment of the first aspect, the drive unit is configured to hermetically seal the bag. In an embodiment of the first aspect, the system also includes an agitator coupled to the drive unit and disposed within the bag. In such an aspect, the agitator can comprise a shaft. One or more impellers can be coupled to the shaft. In another embodiment, the drive unit includes a fluidic motor, an inlet port in flow communication with the fluidic motor, and an outlet port in flow communication with the fluidic motor. The inlet port is configured to receive pressurized driving fluid and the outlet port is configured to exhaust the driving fluid. In such an embodiment, the inlet port and outlet port can be connected to the drive unit inside the bag or outside the bag. In a further embodiment of the first aspect, the drive unit is configured to be driven by a liquid. In such an embodiment, the system can include a fluidic supply system in flow communication with the drive unit, the supply system configured to provide liquid to the drive unit at a controllable flow rate. In a still further embodiment, the drive unit is configured to be driven by a gas. In such an embodiment, the system can include a supply system in flow communication with the drive unit, the supply system configured to provide gas to the drive unit at a controllable flow rate. In another embodiment of the first aspect, the drive unit is configured to rotate when provided with the driving fluid. In such an embodiment, the drive unit can be configured to rotate at less than about 1200 rpm, at less than about 600 rpm, or at less than about 300 rpm. In yet another embodiment, the bag and the drive unit comprise one or more sterilizable plastics. In another embodiment of the first aspect, the system also includes means for stabilizing the system within a bioreactor holding vessel. In an aspect of such an embodiment, the stabilizing means comprises at least one support connected to the bag. In an embodiment comprising an agitator coupled to the drive unit, the bag can comprise a top portion and a bottom portion, and the drive unit can be attached to the bottom portion of the bag with the agitator extending in an upward direction therefrom. In another such embodiment, the drive unit can be attached to the top portion of the bag with the agitator extending in a downward direction therefrom. In such an embodiment, the system can also include a plate connected to the top portion of the bag for stabilizing the bag when the bag is placed in a bioreactor holding vessel. In another such embodiment, the system can include a rigid or semi-rigid support structure connected to the bag.

In a second aspect, a bioreactor system includes a bag comprising an integral fluidic drive unit and an agitator disposed within the bag and operatively connected to the drive unit such that a movement of the drive unit moves the agitator. In an embodiment of such an aspect, the bag comprises a flexible plastic. In another embodiment, the agitator comprises a shaft coupled to at least one impeller.

In a third aspect, a bioreactor system includes a bag and a drive unit in sealed cooperation with the bag. The bag is configured to hold media, the bag having at least one opening. The drive unit is in sealed cooperation with the bag at the at least one opening so as to create a hermetic seal between the drive unit and the bag, the drive unit configured to rotate an agitator coupled to the drive unit and disposed inside the bag without introducing contamination into the bag.

In a fourth aspect, a method of manufacturing a flexible bag bioreactor is provided. The method includes hermetically sealing a fluidic drive unit into a portion of a flexible bag, the drive unit having an agitator that is disposed inside the bag, wherein the drive unit is configured to move the agitator when a driving fluid is introduced into the drive unit.

In a fifth aspect, a method of agitating media contained in a flexible bag of a bioreactor is provided. The bag has a fluidic drive unit connected to a portion of the bag and an agitator coupled to the drive unit and disposed inside the bag. The method includes introducing a fluid into the drive unit to cause a portion of the drive unit to move and moving an agitator through the media using the movement of said portion of the drive unit. In an embodiment of such an aspect, the fluid is substantially sterilized or purified compressed air. In another embodiment, the fluid is substantially sterilized or purified water.

In a sixth aspect, a bioreactor system is provided which includes a sterilized bag for holding media and means for agitating media in the bag, wherein the agitating means is disposed into a portion of a surface of the bag and configured to operate with a provided drive fluid. In an embodiment of such an aspect, the agitating means comprises a fluidic drive unit that is configured to rotate upon introduction of a drive fluid into the drive unit, the system further comprising a sensor for detecting the rate of rotation of the drive unit.

### BRIEF DESCRIPTION OF THE DRAWINGS

FIG. 1 is a side elevation view illustrating a conventional disposable bioreactor system with an external drive unit.

FIG. 2 is a side elevation view illustrating a bioreactor system having an integrated drive unit according to an embodiment.

FIG. 3 is a side elevation view illustrating a bioreactor system with an integrated drive unit according to another embodiment.

FIG. 4 is a side elevation view showing a bioreactor system with an integrated drive unit according to yet another embodiment situated in a bioreactor vessel.

FIG. 5 is a side elevation view showing a bioreactor system having a bottom-mounted integrated drive unit according to a further embodiment of the invention.

FIG. 6 is a side elevation view illustrating a bioreactor system having a multi-bladed agitator.

FIG. 7 is a side elevation view illustrating a bioreactor system having a paddle agitator.

FIG. 8 shows a top plan cross-sectional view of the embodiment shown in FIG. 2, taken along line 8-8 of FIG. 2.

FIG. 9 is a side elevation view illustrating a bioreactor system with a top plate and a drive unit integrally formed with the top plate according to a further embodiment of the invention, situated in a bioreactor vessel.

FIG. 10 is a schematic representation of a bioreactor system according to another embodiment.

FIG. 11 is a process diagram illustrating a method of agitating media in a bioreactor, according to a further embodiment.

### DETAILED DESCRIPTION OF THE PREFERRED EMBODIMENTS

The following detailed description is directed to certain specific embodiments of the invention. However, the invention can be embodied in a multitude of different ways. In this description, reference is made to the drawings wherein like parts are designated with like numerals throughout.

Flexible, sterilized, disposable bioreactors that have sterilizable agitation fluidic driving units have been developed to address a need to avoid contamination from the drive systems used for agitation in glass or steel bioreactors, and address other problems identified in the prior art. A fluidic motor (e.g., a fluid-driven motor), also referred to herein as a fluidic drive unit, can be incorporated or integrated into a portion of a bioreactor “bag” that is configured to contain the medium (for example, for cell growth). Preferably, the bioreactor bag is hermetically sealed with the drive unit at any portion of the drive unit that extends through the bag. The terms “built-in,” “integrated” and “integral,” as used herein to describe two elements of a system, mean more than simply connected; instead, they describe a first element which is attached to a part of a second element to form, for example, a hermetic seal (or connection) or a connection which is not readily detachable and which, in some embodiments, can not be detached without rendering the elements unsuitable for their intended purposes.

Such fluidic drive units typically have no moving parts interacting with the environment outside of the bioreactor bag (other than a driving fluid) and accordingly minimize risk of contaminants entering through the bioreactor bag from a bearing or coupling mechanism. The driving fluid (e.g., a gas or liquid) may be provided to the driving unit at a controlled flow rate. The driving fluid provides a desired driving force that moves an agitator that is connected to the driving unit, and in contact with the media in the bioreactor bag, to provide the desired agitation. The driving fluid can be purified, filtered and/or sterilized to obviate risk of contamination from any interaction with the fluid and the media. In some embodiments the driving fluid is air or water. The air can be from a common compressed air source, but preferably is filtered, sterilized, and/or purified air. The water is preferably distilled water or purified by reverse-osmosis. Typically the fluidic drive units employ a rotational driving means to agitate the medium, but other drive units and agitation devices that are configured to be driven by a fluidic fluid can also be used.

Various embodiments of the invention provide for systems and methods of introducing agitation into a flexible bioreactor bag in a sterile manner, while desirably creating a hydrodynamic environment similar to that of larger, non-disposable reactors. Such systems are easy to handle and are sterile out-of-the-box, so additional cleaning or sterilization is unnecessary. They require little training to operate, yet provide the nutrient mixing capability required for successful cell and tissue cultures. Such disposable bioreactors are equally useful for the production of, for example, chemicals, biopharmaceuticals, cells, microorganisms, plant metabolites, and foods. The bioreactor embodiments described herein can be used for single use bioreactors, stirred tank reactors, and the like. Such reactors have a variety of applications, such as for the production of therapeutic proteins via batch cell culture. For example, these systems can be used to provide for cell growth and antibody production for Chinese Hamster Ovary (“CHO”) and other cell lines.

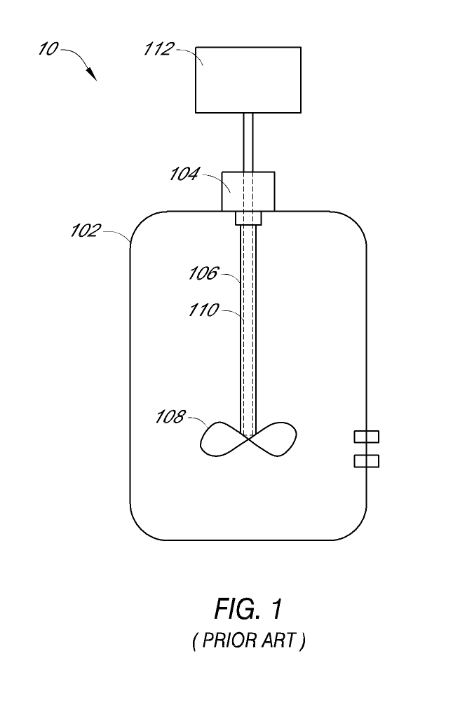

As shown in FIG. 1, a conventional disposable bioreactor system 10 may include a flexible bag 102 and a built-in, disposable rotational assembly 104. The rotational assembly 104 is coupled to a sleeve 106 which terminates in an impeller 108. The sleeve 106 is configured to receive an external shaft 110, which is configured to engage the impeller 108. The rotational assembly 104 and the shaft 110 are driven by an external motor 112 which, as noted in the Background section, introduces an undesirable point of maintenance and expense for users. The introduction of the external shaft 110 through the rotational assembly 104 also creates the unfortunate possibility of introducing contamination into the bag.

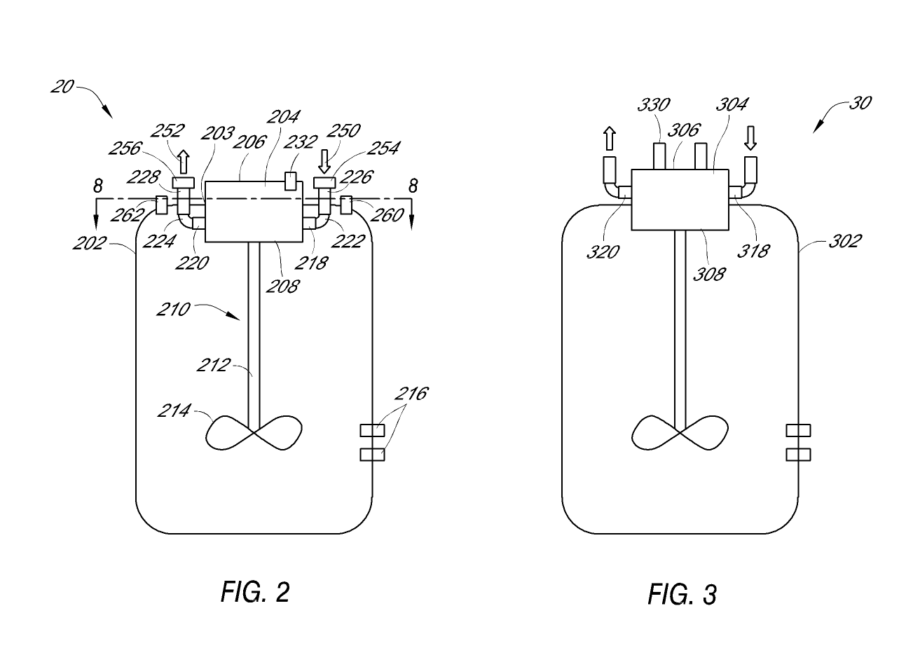

With reference now to FIG. 2, according to some embodiments, a bioreactor assembly 20 generally includes a flexible bag 202 having an opening 203. The assembly 20 also includes a fluidic drive unit 204 and an agitator 210. The drive unit 204 can be driven by a sterile fluid. The sterile fluid can be, for example, a gas (e.g., air) or a liquid, (e.g., distilled or purified water). Because the driving force is provided by a sterile or purified fluid, any leakage that may occur will not contaminate the contents of the bag 202. As shown in FIG. 2, the drive unit 204 may be positioned at least partially in the opening 203 and attached to the bag 202 so as to seal the opening 203. The drive unit 204 may have a first portion 206 disposed outside the bag 202 and a second portion 208 disposed inside the bag 202. The second portion 208 of the drive unit 204 may be operatively coupled to the agitator 210. The agitator 210 may comprise, for example, a shaft 212 and an impeller 214.

### Bioreactor Bag

The bioreactor bag 202 can be a flexible or semi-flexible container configured to hold a fluidic medium (which is referred to herein as “media”). The bioreactor bag 202 is typically easily sterilizable (e.g., by exposing to gamma radiation). In some embodiments, the bioreactor bag 202 includes components, discussed below, which are also sterilizable. The bag 202 may include one or more layer(s) of flexible or semi-flexible material capable of containing media. The material used for manufacturing the bag 202 for a particular application can depend on the specific size, strength and volume requirements for that application. The bioreactor bag 202 can comprise one or more types of plastics or other sterilizable materials, such as, for example, polypropylene or polyvinylidene fluoride (“PVDF”). The bioreactor bag 202 can be manufactured (relatively) inexpensively so that it is disposable. In a bag that includes two or more layers, a first layer may be configured to contain the fluidic media and a second layer may be configured to provide strength to prevent the first layer from rupturing. In some embodiments, the inside surface of the bag 202 may be smooth and provide a sterile environment that can be used for, e.g., culturing cells or other organisms, or for food production. The bioreactor bag 202 may have a capacity of between 100 milliliters and 5000 liters.

The bag 202 may include one or more openings, including opening 203 which can be configured to closely receive and surround the drive unit 204. To maintain a sterile environment within the bag 202, all opening in the bag that allow parts or other components to penetrate the bag 202 are preferably hermetically sealed. The bag 202 may further include one or more ports 216 that facilitate using one or more probes or devices with the bioreactor bag. For example, the ports 216 can be used for collecting a sample, introducing a gas or a fluid into the media, sparging, sensing a condition in the bag 202 (e.g., temperature, pH, dissolved oxygen, or CO2), providing secondary agitation, interaction with an optical sensor and/or a spectrometer, providing heating or cooling, and/or sensing another determinable media characteristic. The bioreactor bag 202 can also include one or more pouches (not shown) which can be used with one or more probes, devices, or the like, or in conjunction with a temperature adjustment system (e.g., a heater or cooler). The bioreactor bag 202 can further include a port 260 configured to allow filling of the bag with media and/or air, as well as a port 262 configured to allow gas to escape during the filling process. In some embodiments, the bioreactor bag 202 can further include a vent filter, a gas overlay port, seals formed in cooperation with bearings and drive unit components, one or more drain ports, and/or an integrated temperature adjustment system (e.g., a fluidic jacket and/or integrated heating system). The bag 202 can provide an entirely disposable alternative to a rigid vessel in a conventional stirred-tank bioreactor where the entire bioreactor bag 202 and its integrated components are disposable.

The bag may further include one or more rigid or semi-rigid supports (not shown) disposed around the sides of the bag, and/or at the top or bottom of the bag. The supports may be configured to support the bag in an upright position when the bag is filled with media. The supports may comprise, for example, one or more ribs, braces, or plates, as well as any combination thereof The supports may be formed from a rigid or semi-rigid plastic. In some embodiments, the bag can include one or more pouches, sleeves, or ring holes to receive inserted supports. In some embodiments, the supports are configured to interact with corresponding structure in a rigid bioreactor vessel to stabilize and/or support the bag 202 within the rigid bioreactor vessel.

### Drive Unit

As shown in FIG. 2, the drive unit 204 may be sealably attached to the bag 202 at the top portion of the bag. In other embodiments the drive unit 204 may be sealably attached on the bottom or the side of the bag. The drive unit 204 may comprise a motor configured to be driven by a fluid such as water or compressed air. Accordingly, as illustrated in FIG. 2, the drive unit 204 may include an inlet port 218 configured to receive driving fluid and an outlet port 220 configured to discharge fluid. As shown in the figure, the inlet port 218 and the outlet port 220 may be located inside the bag 202 on the second portion 208 of the drive unit 204. In this embodiment, the inlet port 218 and the outlet port 220 may be coupled to an inlet tube 222 and an outlet tube 224, respectively. The bag 202 may include two ports 226, 228 through which tubes 222, 224 may pass. The ports 226, 228 may comprise, for example, Pall Kleenpak™ connectors to provide a sterile connection with a drive fluid supply line. The drive unit 204, and all its internal parts, may comprise one or more sterilizable plastics. The entire bioreactor assembly 20, including the drive unit 204, may be disposable. To ensure the integrity of the sterile environment within the bag 202, the openings in the bag 202 for the drive unit 204 and ports 226, 228 are hermetically sealed, e.g., in sealed cooperation with the component that penetrates the bag 202. A flow meter 232 can be provided to monitor the fluid passing through the unit 204 and control the unit's speed. In addition, a filter 254 may be positioned such that a drive fluid flowing through the inlet tube 222 passes through the filter 254 before it enters the drive unit 204. For example, when a gas (e.g., air) is used to drive the unit 404, filter 254 may comprise an air filter to filter particles greater or equal to 0.2 microns. The filter 254 may be disposed outside of the bag 202 (as shown in FIG. 2), or outside of the bag. The filter 254 may also be disposable. The outlet port may also include a similar filter 256.

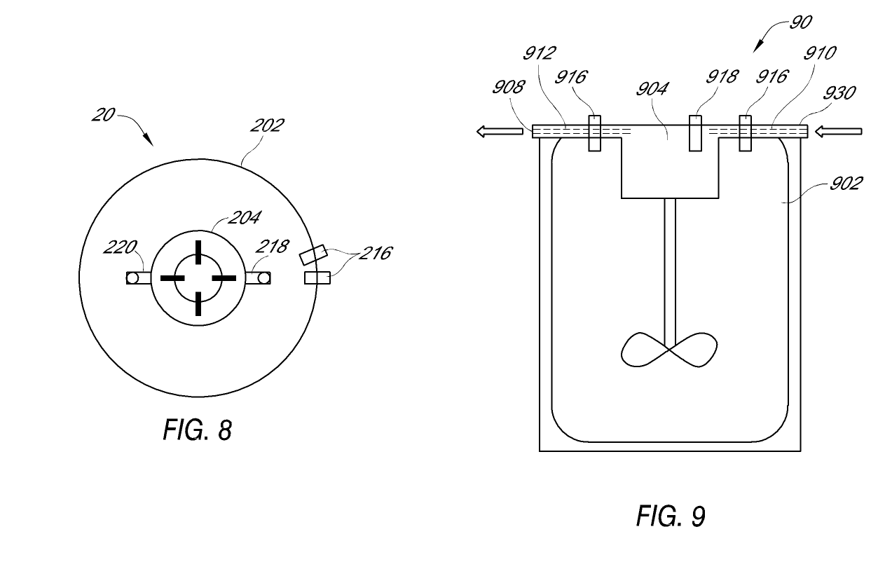

With reference now to FIG. 8, a cross-sectional view of the bioreactor assembly 20 of FIG. 2 is illustrated. As can be seen FIG. 8, the drive unit 204 may comprise, for example, a rotary vane air motor. The vanes can be straight, or curved to provide a larger surface to receive force from the driving fluid. Use of air as a driving force can be advantageous because compressed air is typically available in labs that use bioreactors. In some embodiments, a digital Mass Flow Controller (“MFC”) or other type of flow meter 232 (see FIG. 2) can be provided in the system and used to control the drive unit. Additionally or alternatively, an optical sensor can be employed to sense the rotational rate of the system.

In an embodiment illustrated in FIG. 3, a bioreactor assembly 30 may include a bag 302 and a drive unit 304. A first portion 306 of the drive unit 304 is disposed outside the bag 302, and a second portion 308 disposed inside the bag 302. In the illustrated embodiment, the drive unit 304 comprises an inlet port 318 and an outlet port 320 located on the first portion 306 of the drive unit 304, minimizing the additional ports required in the bag 302. In some embodiments, the inlet port 318 and the outlet port 320 may be disposed on one or more sides of the drive unit 304, for example, illustrated in FIG. 3 on opposite sides of the drive unit 304. Other embodiments may have an inlet and outlet port disposed at the top of the drive unit 304 or on the same side. In still other embodiments, a fluidic inlet port may be disposed inside the bag 202 and a fluidic output port may be disposed outside the bag 202, or vice versa. The drive unit 304 may further comprise pins 330 attached to the first portion 306. The pins 330 may connect to rods (not shown) or other means to stabilize the assembly 30 within a bioreactor vessel.

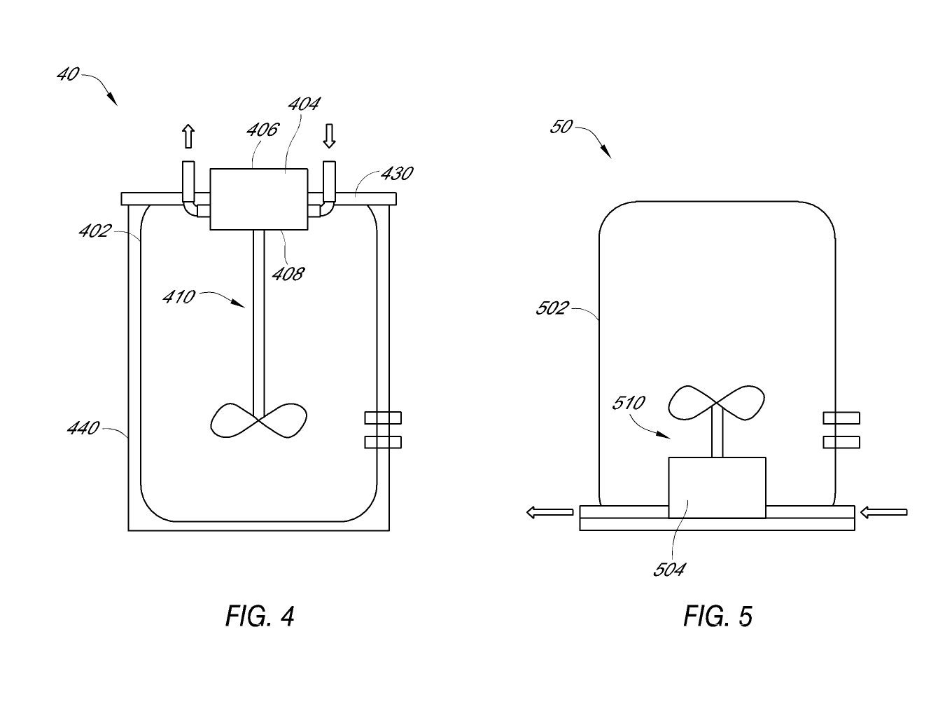

A bioreactor assembly may be supported or stabilized by rigid or semi-rigid structures that mechanically support the bioreactor assembly and/or are attached to the bioreactor assembly. As illustrated in an embodiment show in FIG. 4, a bioreactor assembly 40 may include a bag 402 and a drive unit 404. The assembly 40 may further include a rigid or semi-rigid top plate 430, attached to the top of the bag 402 and disposed around the circumference of the drive unit 404. The top plate 430 may extend laterally beyond the side walls of the bag 402, and may be configured to support and stabilize the bag 402, drive unit 404, and agitator 410 within a bioreactor vessel 440. Any of the bioreactor bags described herein may also include a rigid or semi-rigid top plate 430. The rigid or semi-rigid top plate 430 may comprise any suitable rigid or semi-rigid material, for example, a polymer. The top plate 430 may further include openings or channels configured to allow supply and exhaust of a driving fluid to and from the drive unit 404. The channels may be substantially vertical, substantially horizontal, or disposed on an incline.

Bioreactor assemblies that incorporate one or more of the described aspects can have other configurations. As illustrated in FIG. 5, a bioreactor assembly 50 includes a bag 502, a drive unit 504, and an agitator 510. The drive unit 504 may be sealably attached to a rigid or semi-rigid plate 532 at the bottom of the bag 502. The agitator 510 may extend vertically (or at an angle) from the drive unit 504. As shown by a comparison of FIGS. 4 and 5, a shorter agitator 510 may be used with the bottom-mounted drive unit 504 (FIG. 5), as compared with the agitator 410 of the top-mounted drive unit 404 (FIG. 4) to provide agitation at the same location within the bag 502 as provided in bag 402. Embodiments having a bottom-mounted drive unit may also include a rigid or semi-rigid top plate for support, as described above.

In still another embodiment, as shown in FIG. 9, a bioreactor assembly 90 includes a bag 902 and a drive unit 904, along with a rigid or semi-rigid plate 930 that is connected to the bag 902. The drive unit 904 may be integral with plate 930. The plate 930 may include an inlet port 906 and an outlet port 908 configured to allow fluid communication with the drive unit 904 via channels 910, 912. The channels 910, 912 allow the supply and exhaust of a driving fluid through the sides of the plate 930 and to and from the drive unit 904. In one alternative embodiment (not shown) the driving fluid inlet and outlet ports are disposed on the top of the plate 930. As shown in the figure, the plate 930 may include ports 916 configured to allow the sterile introduction of probes or sensors inside the bag 902. The plate 930 may further include additional ports, such as aeration ports, for example. The drive unit 904 can be provided with one or more optical sensors 918 configured to sense the effective rotational rate or speed of the drive unit 904.

### Seal

Referring back to FIG. 2, the opening 203 in the bag 202 may be hermetically sealed around the drive unit 204. The seal between the drive unit 204 and the opening 203 is a stationary seal which does not introduce a risk of leakage or contamination. Such a seal may be achieved using any suitable means, including, but not limited to, glue, heat sealing, o-rings or v-seals (an elastomeric seal having a roughly v-shaped cross section, configured to seal against a counterface). Alternatively, in some embodiments of the invention, the second portion 208 of the drive unit 204 may be molded into the wall of the bag 202 during fabrication. In still other embodiments, an airtight seal may be achieved via a casing which surrounds the drive unit 204 and seals with the opening 203.

### Agitator

With continued reference to FIG. 2, the agitator 210 may be coupled to the second portion 208 of the drive unit 204. The agitator 210 can be any sized or shaped device capable of agitating or mixing the contents of a bioreactor. The agitator 210 may agitate the contents of the system by stirring or other mechanical motion. Depending on the application, and agitation requirements of the fermentation process, the size and shape of the agitator 210 may vary.

Although the illustrated agitator 210 is disposed along a vertical axis, embodiments of the invention also include agitators which may enter the bag 202 at an angle. Additionally, the agitator 210 may be angled with respect to the drive unit 204.

As illustrated in the figure, the agitator 210 may comprise a shaft 212 and an impeller 214. The agitator rotation speed may be controlled by adjusting the driving fluid flow rate flowing through the drive unit 204.

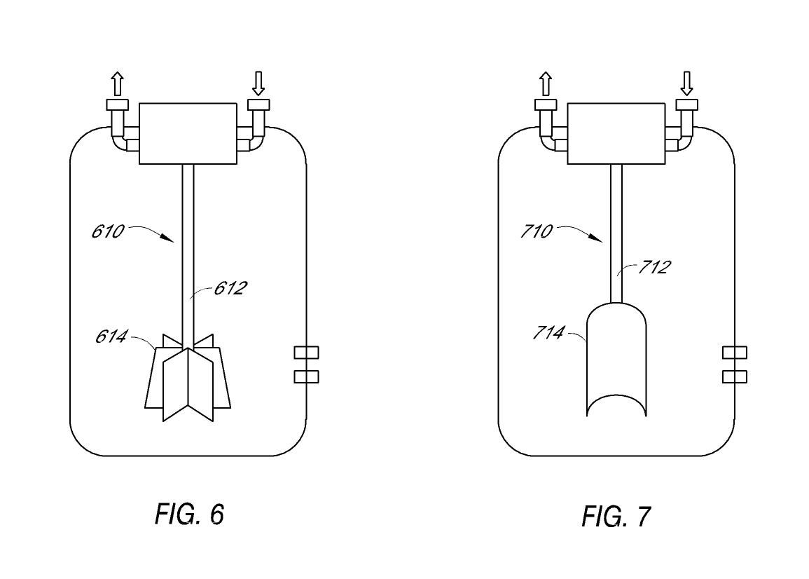

The impeller of the instant invention includes, but is not limited to, a Rushton, a marine, a hydrofoil, a pitched blade, and any other commercially available impeller. Some embodiments include two or more impellors. FIG. 6, for example, illustrates an embodiment comprising an agitator 610 having a shaft 612 and a four-blade axial impeller 614. Referring now to FIG. 7, embodiments of the invention may alternatively include an agitator 710 comprising a shaft 712 and a paddle 714. In some embodiments, the agitator 710 may be configured to mix the contents of the reactor system using an oscillating or back-and-forth motion.

Embodiments of the invention may also include one or more sensors to obtain direct feedback on the rate of the rotation of the agitator. These sensors may, for example, be optical sensors configured to sense rate of rotation without introducing any contamination inside the bag. In one embodiment, an optically sensed target is placed at one or more locations on a rotational element within the drive unit, and the rotation rate is derived from measuring (or counting) the number of times the target passes an optical sensor.

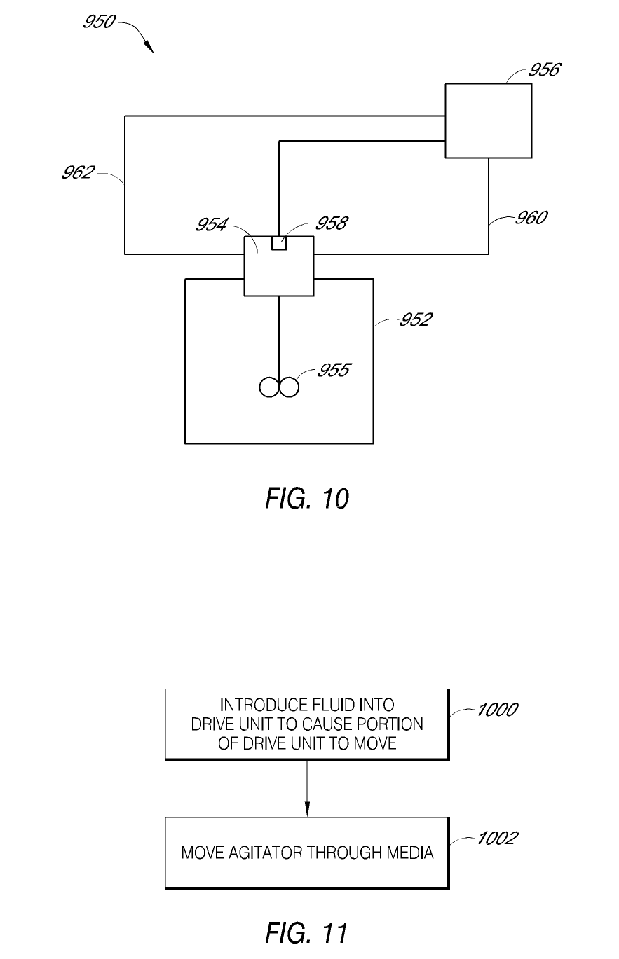

The rate of rotation of the agitator can also be determined indirectly if the rotation corresponds consistently with the driving fluid flow rate. In this case, a flow rate sensor may determine the driving fluid flow rate and this along with information correlating the flow rate and rotation rate can be used to determine the agitation level (e.g., number of RPM's of agitation). FIG. 10 schematically illustrates an embodiment of a bioreactor system 950 which includes a bag 952 having an integrated drive unit 954. The drive unit 954 is coupled to an agitator 955. The system 950 includes a fluid supply unit 956 which is configured to supply pressurized fluid (e.g., air or water) to the drive unit 954 at a controllable flow rate. The system additionally includes one or more sensors 958 configured to sense an agitation parameter of the system 950 and provide feedback to the fluid supply unit 956. The agitation parameter can be, for example, a flow rate of the fluid into or out of the drive unit 954, in which case the sensor 958 can be a flow rate sensor. The agitation parameter can also be a rotation rate of the drive unit 954 or agitator 955, in which case the sensor 958 can be an optical sensor. One or more sensors can be provided in the drive unit 954, as shown in the figure, or can be provided in the fluid inlet path 960, the fluid outlet path 962, or any other suitable location inside or outside of the bag 952. A bioreactor control system (not shown) can be configured to control the fluid supply unit 954. In one embodiment, the bioreactor control system is a BioNet® system, available from Broadley-James Corporation, Irvine, Calif.

### Certain Methods of Use

In other embodiments, the invention comprises a method of agitating the contents of a reactor system. Referring once again to FIG. 2, embodiments of the invention include coupling a flexible bag 202 with the fluidic drive unit 204 so as to seal the opening 203 of the bag 202. The agitator 210 is also coupled to the drive unit 204 at the second portion 208 of the bag 202. Next, a pressurized fluid is introduced to the drive unit 204 along inlet path 250 through inlet port 218. The fluid is expelled from the drive unit 204 through outlet port 220, along outlet path 252. The pressurized fluid generates rotational or other motion in the drive unit 204, causing the agitator 210 to rotate or move through the contents of the bag 202. Embodiments of the invention may utilize any suitable fluid to drive the drive unit 204. For example, a filtered sterile gas (e.g., air or nitrogen) or a sterile liquid (e.g. purified sterile water, or a sterile electrolyte solution) can be used to eliminate the possibility of introducing contamination into the bag 202 should any leakage occur.

With reference now to FIG. 11, according to another embodiment, a method of agitating media contained in a flexible bag of a bioreactor is illustrated. The bag has a fluidic drive unit connected to a portion of the bag and an agitator coupled to the drive unit. The agitator is disposed inside the bag. At step 1000, a fluid is introduces into the drive unit to cause a portion of the drive unit to move. At step 1002, the movement of the portion of the drive unit is used to move an agitator through the media.

A method of manufacturing a flexible bioreactor bag is also provided. According to an embodiment, the method includes hermetically sealing a fluidic drive unit into a portion of a flexible bag. The drive unit has an agitator that is disposed inside the bag. The drive unit is configured to move the agitator when a driving fluid is introduced into the drive unit.

Referring again to FIG. 2, it can be seen that embodiments of the invention provide a shaft-stirred system without any axial penetration of the bag 202. Because the drive unit 204 is hermetically sealed with the bag 202, these and other embodiments form a closed system that advantageously eliminates the possibility of introducing external contaminants into the bag.

As will be apparent to one of skill in the art, embodiments of the invention also allow for smaller-scale development reactor systems, for example, systems having a three to 20 liter capacity, to simulate the hydrodynamic environments of larger-scale production systems, while avoiding the cumbersome cleaning requirements of conventional systems.

Embodiments of the invention also desirably eliminate the need for external electric motors, thereby eliminating a point of maintenance and expense for users. Furthermore, providing a fluidic motor advantageously allows a user to easily reverse the direction of motion of the motor, by simply attaching the fluid supply line to a different port.

Various modifications to these examples may be readily apparent to those skilled in the art, and the generic principles defined herein may be applied to other examples without departing from the spirit or scope of the novel aspects described herein. Thus, the scope of the disclosure is not intended to be limited to the examples shown herein but is to be accorded the widest scope consistent with the principles and novel features disclosed herein.

What is claimed is:

1. A bioreactor system comprising:
a flexible bag; and
a fluid activated drive unit in sealed cooperation with the bag.

2. The bioreactor system of claim 1, wherein the drive unit is configured to hermetically seal the bag.

3. The bioreactor system of claim 1, further comprising an agitator coupled to the drive unit and disposed within the bag.

4. The bioreactor system of claim 2, wherein the agitator comprises a shaft.

5. The bioreactor system of claim 4, further comprising at least one impeller coupled to the shaft.

6. The bioreactor system of claim 1, wherein the drive unit comprises:
a fluidic motor;
an inlet port in flow communication with the fluidic motor, the inlet port configured to receive pressurized driving fluid; and
an outlet port in flow communication with the fluidic motor, the outlet port configured to exhaust the driving fluid.

7. The bioreactor system of claim 6, wherein the inlet port and outlet port are connected to the drive unit inside the bag.

8. The bioreactor system of claim 6, wherein the inlet port and outlet port are connected to the drive unit outside the bag.

9. The bioreactor system of claim 1, wherein the drive unit is configured to be driven by a liquid.

10. The bioreactor system of claim 9, further comprising a fluidic supply system in flow communication with the drive unit, the supply system configured to provide liquid to the drive unit at a controllable flow rate.

11. The bioreactor system of claim 1, wherein the drive unit is configured to be driven by a gas.

12. The bioreactor system of claim 11, further comprising a supply system in flow communication with the drive unit, the supply system configured to provide gas to the drive unit at a controllable flow rate.

13. The bioreactor system of claim 1, wherein the drive unit is configured to rotate when provided with the driving fluid.

14. The bioreactor system of claim 13, wherein the drive unit is configured to rotate at less than about 1200 rpm.

15. The bioreactor system of claim 13, wherein the drive unit is configured to rotate at less than about 600 rpm.

16. The bioreactor system of claim 13, wherein the drive unit is configured to rotate at less than about 300 rpm.

17. The bioreactor system of claim 1, wherein the bag and the drive unit comprise one or more sterilizable plastics.

18. The bioreactor system of claim 1, further comprising means for stabilizing the system within a bioreactor holding vessel.

19. The bioreactor system of claim 18, wherein the stabilizing means comprises at least one support connected to the bag.

20. The bioreactor system of claim 3, wherein the bag comprises a top portion and a bottom portion, and wherein the drive unit is attached to the bottom portion of the bag and the agitator extends in an upward direction therefrom.

21. The bioreactor system of claim 3, wherein the bag comprises a top portion and a bottom portion, and wherein the drive unit is attached to the top portion of the bag and the agitator extends in a downward direction therefrom.

22. The bioreactor system of claim 21, further comprising a plate connected to the top portion of the bag for stabilizing the bag when the bag is placed in a bioreactor holding vessel.

23. The bioreactor system of claim 22, further comprising a rigid or semi-rigid support structure connected to the bag.

24. A bioreactor system, comprising:
a bag comprising an integral fluidic drive unit; and
an agitator disposed within the bag and operatively connected to the drive unit such that a movement of the drive unit moves the agitator.

25. The bioreactor system of claim 24, wherein the bag comprises a flexible plastic.

26. The bioreactor system of claim 24, wherein the agitator comprises a shaft coupled to at least one impeller.

27. A bioreactor system comprising:
a bag configured to hold media, the bag having at least one opening; and
a drive unit in sealed cooperation with the bag at the at least one opening so as to create a hermetic seal between the drive unit and the bag, the drive unit configured to rotate an agitator coupled to the drive unit and disposed inside the bag without introducing contamination into the bag.

28. A method of manufacturing a flexible bag bioreactor, the method comprising hermetically sealing a fluidic drive unit into a portion of a flexible bag, the drive unit having an agitator that is disposed inside the bag, wherein the drive unit is configured to move the agitator when a driving fluid is introduced into the drive unit.

29. A method of agitating media contained in a flexible bag of a bioreactor, the bag having a fluidic drive unit connected to a portion of the bag and an agitator coupled to the drive unit and disposed inside the bag, the method comprising:
introducing a fluid into the drive unit to cause a portion of the drive unit to move; and
moving an agitator through the media using the movement of said portion of the drive unit.

30. The method of claim 29, wherein the fluid is substantially sterilized or purified compressed air.

31. The method of claim 29, wherein the fluid is substantially purified water.

32. A bioreactor system, comprising:
a sterilized bag for holding media; and
means for agitating media in the bag, wherein the agitating means is disposed into a portion of a surface of the bag and configured to operate with a provided drive fluid.

33. The bioreactor system of claim 32, wherein the agitating means comprises a fluidic drive unit that is configured to rotate upon introduction of a drive fluid into the drive unit, the system further comprising a sensor for detecting the rate of rotation of the drive unit.
